(19) United States
(12) Patent Application Publication
(10) Pub. No.: US 2020/0264098 A1
(43) Pub. Date: Aug. 20, 2020

(54) SYSTEM, COMPOSITION AND METHOD FOR THE DETECTION OF SPECTRAL BIOMARKERS OF A CONDITION AND PATTERNS FROM STOOL SAMPLES

(71) Applicant: ClinicAI, Inc., San Francisco, CA (US)

(72) Inventor: Chun-Hao Huang, San Francisco, CA (US)

(21) Appl. No.: 16/869,567

(22) Filed: May 7, 2020

Related U.S. Application Data
(63) Continuation of application No. PCT/US2018/059275, filed on Nov. 5, 2018.
(60) Provisional application No. 62/584,561, filed on Nov. 10, 2017.

(51) Int. Cl. G01N 21/31; G01N 33/574; G06T 7/00

(52) U.S. Cl. CPC G01N 21/31; G01N 33/57419; G06T 2207/20081; G06T 2207/30032; G06T 7/0012

(57) ABSTRACT

A system, composition and method detect diseases using a method for identifying spectral biomarkers and patterns from stool samples. In one embodiment, the system, composition and method may provide a non-invasive method for detecting colorectal cancer and precancerous polyps comprises subjecting stool samples from cancerous and non-cancerous subjects to hyperspectral spectroscopy and wherein differences in spectra indicates cancer, or assesses risk of development thereof. The system, composition and method may also include a method for identifying spectral biomarkers and patterns from stool samples from cancerous, precancerous and inflammatory bowel disease subjects.

### PRIORITY CLAIM/RELATED APPLICATIONS

This application claims the benefit under 35 USC 119(e) to U.S. Provisional Patent Application Ser. No. 62/584,561 filed Nov. 10, 2017 and entitled “Composition and Method for the Detection of Colorectal Cancer from Stool Samples”, the entirety of which is incorporated herein by reference.

### FIELD

This application relates generally to a system, compositions and methods for disease detection, as well as method for identifying spectral biomarkers and patterns from stool samples.

### BACKGROUND

There are many conditions of the human body, such as various diseases, in which it is possible to detect spectral biomarkers of a condition, determine patterns and be able to detect the condition. Most of the existing solutions are invasive and require a needle prick or blood to be drawn to be able to detect the condition. As example of a condition that may be detected is colorectal cancer.

Colorectal cancer is the third most common cancer worldwide and also the second leading cause of cancer related deaths in developed countries yet there remain few effective therapies for treating advanced disease. Although early diagnosis could save the lives of colorectal cancer patients, it has been challenging to detect tumors at early stages due to invasiveness and costliness of diagnostic methods such as colonoscopy. There is therefore a need to develop a non-invasive screening method for early diagnosis of colorectal cancer.

Recently, colorectal cancer screening kit, such as stool kits for blood and DNA (Cologuard) have appeared on the market, but those screening kits all need stool collection, sample preparation and submission to a lab for analysis of blood chemistry or dna to detect colorectal cancer or a risk of colorectal cancer. It would be desirable to be able to do detection and screening without stool collection or sample preparation.

Previous studies using spectroscopy for fecal analysis suggest that infrared or magnetic resonance spectroscopy of buffer suspension of stool samples can be used as a method of detecting colorectal cancer (WO2005017501A1). However, the potential of direct analysis of stool samples without mixing them with buffer to extract the supernatant for analysis has been unclear.

Recent developments in spectroscopy, such as hyperspectral spectroscopy, allow us to examine visible and invisible absorptive spectrum in human specimens across different wavelength bands. Although hyperspectral spectroscopy has been explored for various applications including disease diagnosis and image-guided surgery, the use of hyperspectral spectroscopy for direct analysis of stool samples as a non-invasive method of detecting colorectal cancer has not been explored.

### SUMMARY

One aspect of the disclosure relates to a system, apparatus and method for detecting colorectal cancer and precancerous polyps from stool samples. The method comprises subjecting stool samples from cancerous and non-cancerous subjects to hyperspectral spectroscopy and wherein differences in spectra indicates cancer, or assesses risk of development thereof. In another aspect, the method may be used for identifying spectral biomarkers and patterns of a condition from stool samples, wherein the condition is selected from the group comprising: colorectal cancer, precancerous polyps, gastrointestinal diseases, inflammatory pain, chronic disease, cardiovascular diseases, cardiac hypertrophy, heart failure and cancer. The cancer may be carcinoma, sarcoma, melanoma, germ cell tumor, lymphomas or leukemia.

In some embodiments, the hyperspectral spectrometer is selected from the group comprising: spectral range between 200 to 11,111 nm, spectral range including Ultraviolet (UV) and visible spectrum and Near-infrared (NIR), spectral resolution between 1 to 10 μm/pixel, spectral resolution (Full Width at Half Maximum) between 0.3 nm to 1.5 nm. The hyperspectral spectrometer may use a Si (Silicon) charge-coupled device (CCD) detector, charge-coupled device (CCD) detector, HgCdTe (Mercury Cadmium Telluride) detector, intensified charge-coupled device (ICCD) detector, InGaAs (Indium Gallium Arsenide) detector, focal-plane array (FPA) detector, filter wheel dispersive device, prism dispersive device, Liquid crystal tunable filters (LCTF) dispersive device, acousto-optic tunable filter (AOTF) dispersive device, grating dispersive device, computer generated holographic (CGH) dispersive device, prism-grating-prism (PGP) dispersive device, staring acquisition mode, pushbroom acquisition mode, Fourier-transform infrared spectroscopy (FTIR) acquisition mode, snapshot acquisition mode, reflectance measurement mode, fluorescence and reflectance measurement mode, transmission measurement mode, fluorescence measurement mode.

In another aspect, a composition for detecting colorectal cancer and precancerous polyps from stool samples is disclosed. The composition comprises: a hyperspectral spectrometer and a statistic method or a computer algorithm to compare the resulting hyperspectral spectrum of stool from non-cancerous and colorectal cancer subjects, and identify differences in spectra being indicative of colorectal cancer and precancerous polyps.

In another aspect, the application relates to a method for detecting colorectal cancer from stool samples without mixing the samples with buffer to extract the supernatant for analysis. The stool samples are directly subjected to hyperspectral spectroscopy, and can be used for other spectroscopy including infrared and near-infrared spectroscopy.

In another aspect, the application relates to a method for identifying novel spectral biomarkers and patterns from stool samples from a condition. The method comprises the steps of: (a) collecting stool samples from subjects with different conditions; (b) analyzing the stool samples by hyperspectral spectroscopy; (c) classifying spectra from stool samples from different subjects; (d) developing computer algorithms; and (e) identifying spectral biomarkers and patterns. The condition is selected from the group comprising: colorectal cancer, precancerous polyps, gastrointestinal diseases, inflammatory pain, chronic disease, cardiovascular diseases, cardiac hypertrophy, heart failure, cancer. The cancer may be carcinoma, sarcoma, melanoma, germ cell tumor, lymphomas or leukemia. In other embodiments, the spectral biomarkers and patterns is in the range between 200 to 11,111 nm. In other embodiments, the spectral biomarkers and patterns is in the range spectral range including Ultraviolet (UV) and visible spectrum and Near-infrared (NIR).

### BRIEF DESCRIPTION OF THE DRAWINGS

FIG. 1 illustrates an example of an embodiment of a system that may use imaging, such as hyperspectral spectrometer, to detect a condition of a human being from a stool sample;

FIGS. 2 and 3 illustrate more details of portions of the system shown in FIG. 1;

FIG. 4 illustrates a method for detecting a condition based on stool samples;

FIG. 5 illustrates an example of hyperspectral imaging analysis that can identify conditions, including colorectal cancer or polyps from the spectral data from the stool samples;

FIG. 6 illustrates examples of the results from the hyperspectral imaging analysis of FIG. 5;

FIG. 7 illustrates an example of single point hyperspectral spectrometer analysis identifies the stool samples from colorectal cancer;

FIGS. 8 and 9 illustrate examples of the results from the hyperspectral spectrometer analysis of FIG. 7;

FIG. 10 illustrates the hyperspectral spectrometer analysis for late stage colorectal cancer and healthy subjects in humans based on human stool;

FIG. 11 illustrates the hyperspectral spectrometer analysis for polyps and early stage colorectal cancer and healthy subjects in humans based on human stool;

FIG. 12 illustrates an example of a first embodiment of the stool sample sensor of the system in FIG. 1;

FIG. 13 illustrates the stool sample sensor of FIG. 12 mounted on a toilet;

FIGS. 14-16 illustrate an example of a second embodiment of the stool sample sensor of the system in FIG. 1 and the stool sample sensor mounted on a toilet;

FIG. 17 illustrates more details of the core device 120 and imaging and sensing device 122 that are part of the stool sample sensor 14;

FIG. 18 illustrates an example of the spectra generated by the image sensor;

FIG. 19 illustrates an example of the user interface for a user generated by the system in FIG. 1; and

FIG. 20 illustrates an example of the user interface for a doctor generated by the system in FIG. 1.

### DETAILED DESCRIPTION OF ONE OR MORE EMBODIMENTS

Some modes for carrying out the disclosed system and method are presented in terms of its exemplary embodiments, herein discussed below. However, the disclosed system and method are not limited to the described embodiment and a person skilled in the art will appreciate that many other embodiments are possible without deviating from the basic concept of the disclosed system and method, and that any such work around will also fall under scope of this application. It is envisioned that other styles and configurations of the disclosed system and method can be easily incorporated into the teachings of the disclosure, and only one particular configuration shall be shown and described for purposes of clarity and disclosure and not by way of limitation of scope. For purposes of illustration, an embodiment of the system and method that obtains spectral images of stool samples for the purpose of detection of colorectal cancer and precancerous polyps are described although the disclosed system and method may more generally be used to gather and identify novel spectral biomarkers and patterns of various conditions from stool samples.

Unless defined otherwise, all technical and scientific terms used herein have the same meanings as commonly understood by one of skill in the art to which the disclosed method and compositions belong. It must be noted that as used herein and in the appended claims, the singular forms “a,” “an,” and “the” include plural reference unless the context clearly dictates otherwise.

As used herein, the terms “detect” and “detection” refer to the identification of one or more symptoms associated with a condition, such as colorectal cancer and diagnosis of the one or more symptoms associated with condition, such as colorectal cancer. The individual (also referred to as “patient” or “subject”) being treated may be a fetus, infant, child, adolescent, or adult human with any condition. The detected condition may include one or more of colorectal cancer, precancerous polyps, gastrointestinal diseases, inflammatory pain, chronic disease, cardiovascular diseases, cardiac hypertrophy, heart failure and cancer.

As used herein the term “cancer” refers to any of the various malignant neoplasms characterized by the proliferation of cells that have the capability to invade surrounding tissue and/or metastasize to new colonization sites, including but not limited to carcinomas, leukemia, lymphoma, sarcomas, melanoma and germ cell tumors. A representative but non-limiting list of cancers that may be treated or identified using the disclosed system and method may include: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “leukemia” refers to broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which arises from transformed cells of mesenchymal origin. Sarcomas are malignant tumors of the connective tissue and are generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphomas (e.g., Non-Hodgkin Lymphoma), immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

As used herein, the term “hyperspectral spectroscopy” refers to spectrometers or sensors that collect and analyze spectral range between 200 to 11,111 nm, including Ultraviolet (UV) and visible spectrum and Near-infrared (NIR). Examples of hyperspectral spectroscopy include, but are not limited to, the hyperspectral spectrometer selected from the group comprising: spectral range between 200 to 11,111 nm, spectral range including Ultraviolet (UV) and visible spectrum and Near-infrared (NIR), spectral resolution between 1 to 10 μm/pixel, spectral resolution (Full Width at Half Maximum) between 0.3 nm to 1.5 nm. Si (Silicon) charge-coupled device (CCD) detector, charge-coupled device (CCD) detector, HgCdTe (Mercury Cadmium Telluride) detector, intensified charge-coupled device (ICCD) detector, InGaAs (Indium Gallium Arsenide) detector, focal-plane array (FPA) detector, filter wheel dispersive device, prism dispersive device, Liquid crystal tunable filters (LCTF) dispersive device, acousto-optic tunable filter (AOTF) dispersive device, grating dispersive device, computer generated holographic (CGH) dispersive device, prism-grating-prism (PGP) dispersive device, staring acquisition mode, pushbroom acquisition mode, Fourier-transform infrared spectroscopy (FTIR) acquisition mode, snapshot acquisition mode, reflectance measurement mode, fluorescence and reflectance measurement mode, transmission measurement mode, fluorescence measurement mode.

As used herein, the terms “subject,” “individual,” and “animal” are used interchangeably herein to refer to a vertebrate, preferably a mammal. The term “mammal” or “mammalian” includes, but is not limited to, murines, simians, humans, farm animals, sport animals, and pets. Tissues, cells and their progeny of a biological entity obtained in vivo or cultured in vitro are also encompassed.

### EXEMPLARY EMBODIMENTS OF THE SYSTEM

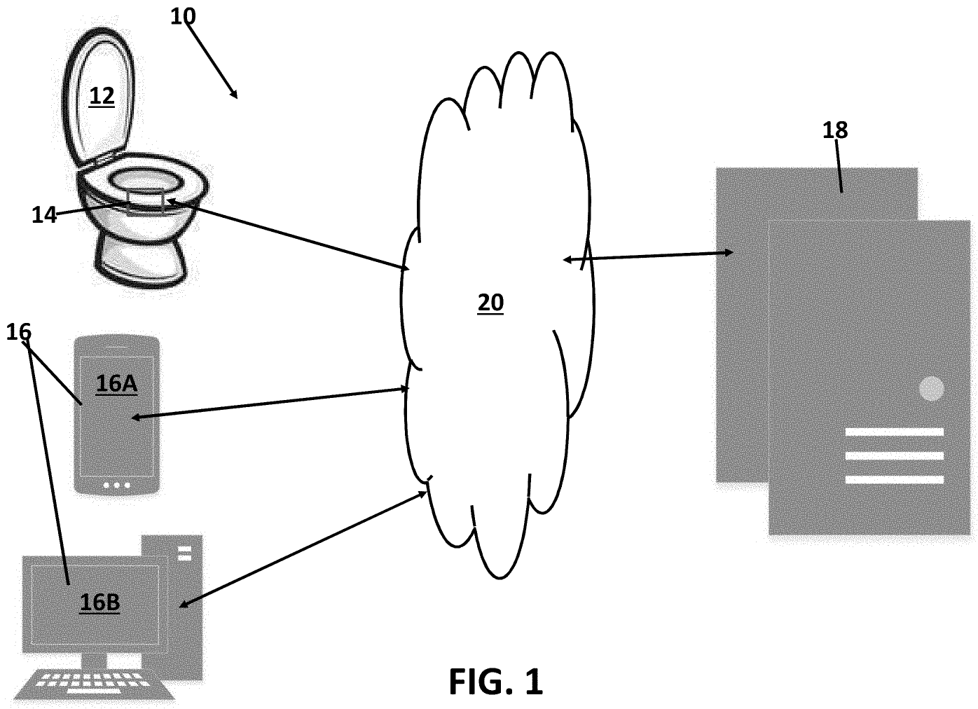

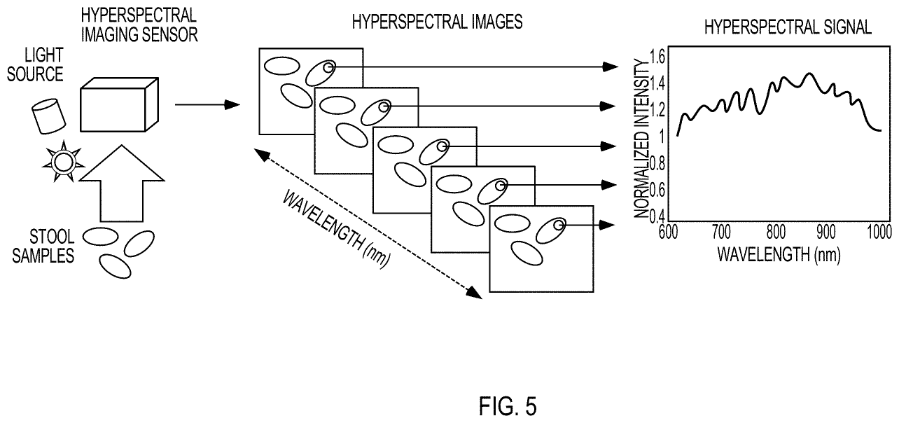

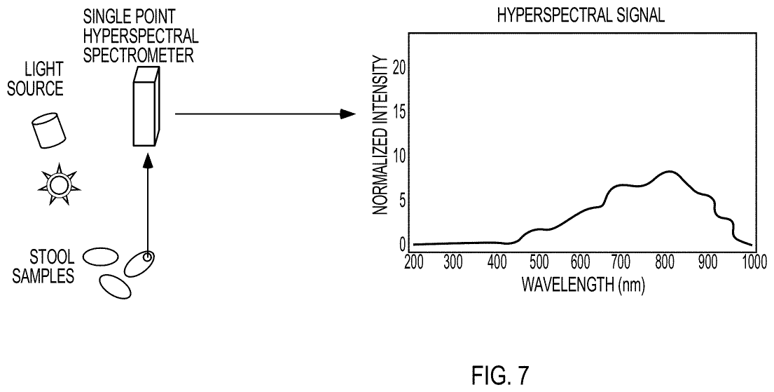

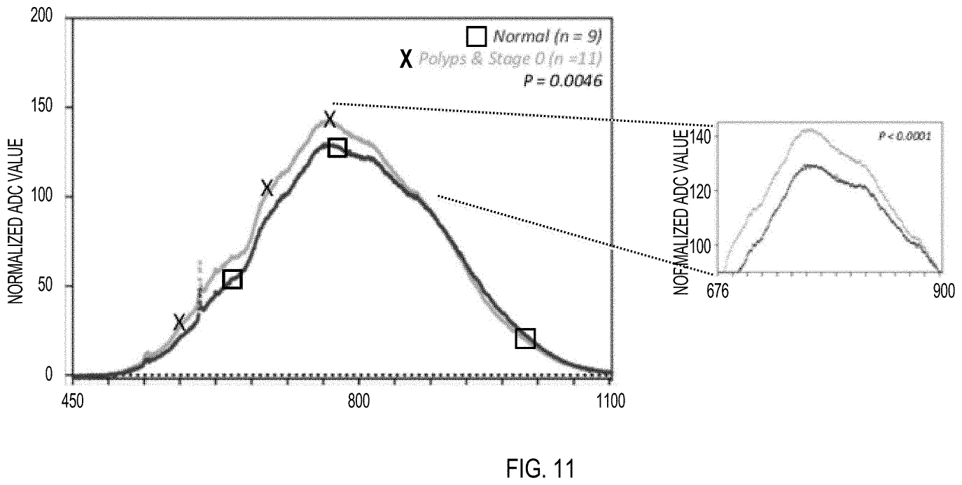

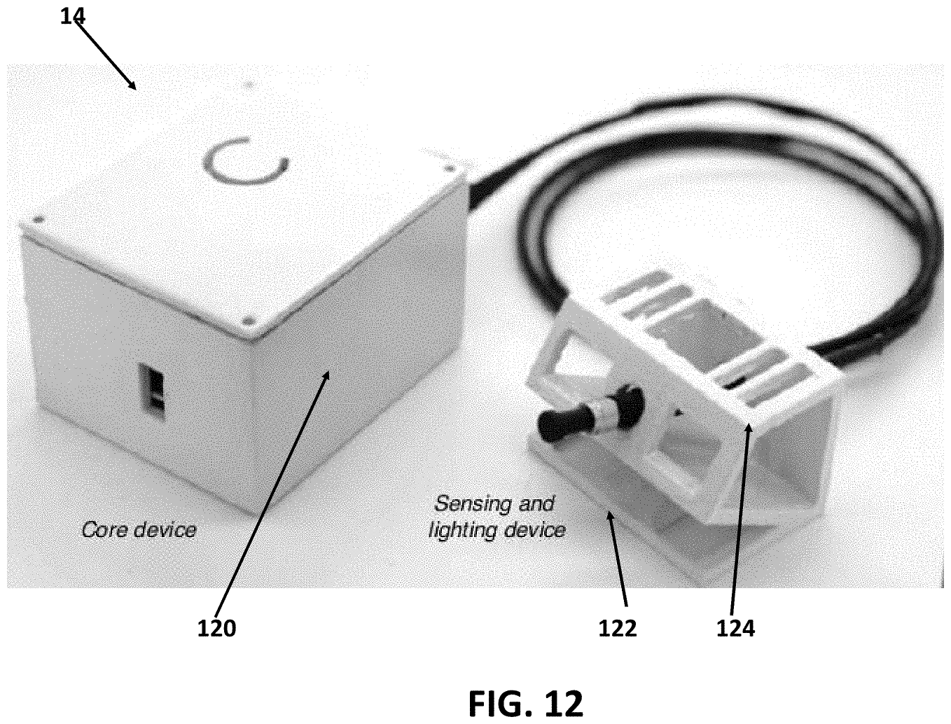

FIG. 1 illustrates an example of an embodiment of a system 10 that may use imaging, such as hyperspectral spectrometer, to detect a condition of a human being from a stool sample. The system 10 may include a typical toilet 12 onto which a stool sample sensor 14 may be mounted/placed wherein the stool sample sensor 14 may generate spectral biomarkers of a condition, such as colorectal cancer in one embodiment. The stool sample sensor 14 may be an optical sensor for various types, including a hyperspectral spectrometer camera (an example of which is shown in FIG. 5), a single point hyperspectral spectrometer (an example of which is shown in FIG. 7) or any other optical sensors that are capable of generating a set of spectral biomarkers at the range of wavelengths disclosed herein. The stool sample sensor 14 may generate the set of spectral biomarkers of one or more different conditions when a subject defecates in the toilet which avoids the subject have to collect the stool sample (like has to be done for the screening kits or does not require the stool samples to the mixed with another substance like known systems before the analysis of the stool sample is performed. Furthermore, since the stool sample sensor 14 is inside/adjacent the toilet, the condition detection is performed in situ (instead of having to send the sample to a lab) and in real time (without having to wait for the sample to arrive at the lab and be tested by the lab. The stool sample sensor 14 may generate the set of spectral biomarkers that may be communicated to a computing device 16 of the user or to a backend system 18 over a communication path 20. The backend system 18, the computing device 16 and the stool sample sensor 14 may each perform part of the analysis (described below in more detail) by which the system identifies symptoms and a condition, like colorectal cancer, from the set of spectral biomarkers for the particular subject gathered/generated by the stool sample sensor 14. In one embodiment, the system collects a signal every 0.3-1 nm wavelength. Further details of the stool sample sensor 14 are provided below with reference to FIGS. 12-17 that show several different embodiments of the stool sample sensor 14. In other embodiments, the stool sample sensor 14 may be used separate from a toilet, but with the data analytics, such as in a hospital setting.

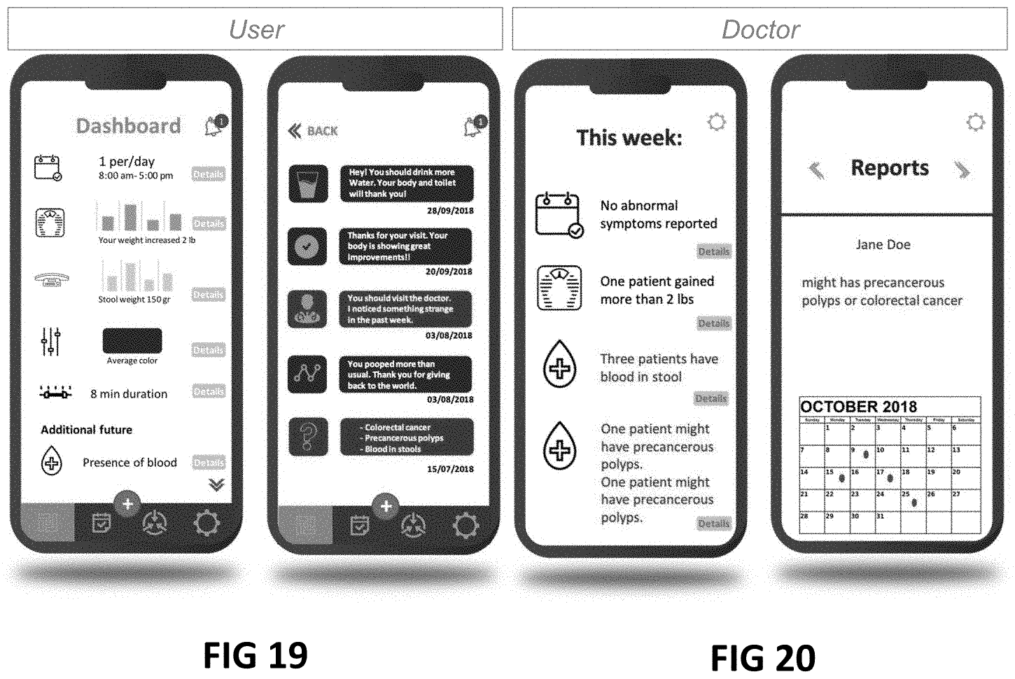

Each computing device 16 may be a processor based device with memory, a display and wired or wireless connectivity circuits that allow the computing device 16 to communicate with the stool sample sensor 14 and/or the backend system 18 and interact/exchange data with the stool sample sensor 14 and/or the backend system 18. For example, the computing device 16 may exchange data with the backend system 18 and receive graphical or visual data showing the results of the analysis of the set of data from the stool sample sensor 14. The system may have multiple computing devices, such as a smartphone device 16A and the personal computer 16B as shown in FIG. 1, and each computing device may be a smartphone device, such an Apple iPhone product or an Android OS based system, a laptop computer, a tablet computer, a personal computer, a terminal device and the like. Each computing device 16 may have an application, a web app or a mobile app that is executed by the processor of the computing device 16 that may visually display information to a user where examples of the user interfaces for a patient/subject are shown in FIG. 19 and an example of the user interfaces for a doctor are shown in FIG. 20. The communications path 20 may include one or more wired or wireless networks/systems that allow the various devices and the backend 20 to connect and communicate with each other using a known data and transfer protocol. The backend system 18 may be implemented as one or more known computing resources that perform the novel processing for the detection of a condition based on the spectral data as described below. The one or more known computing resources may be cloud servers, server computers, blade servers, application servers, database servers and the like.

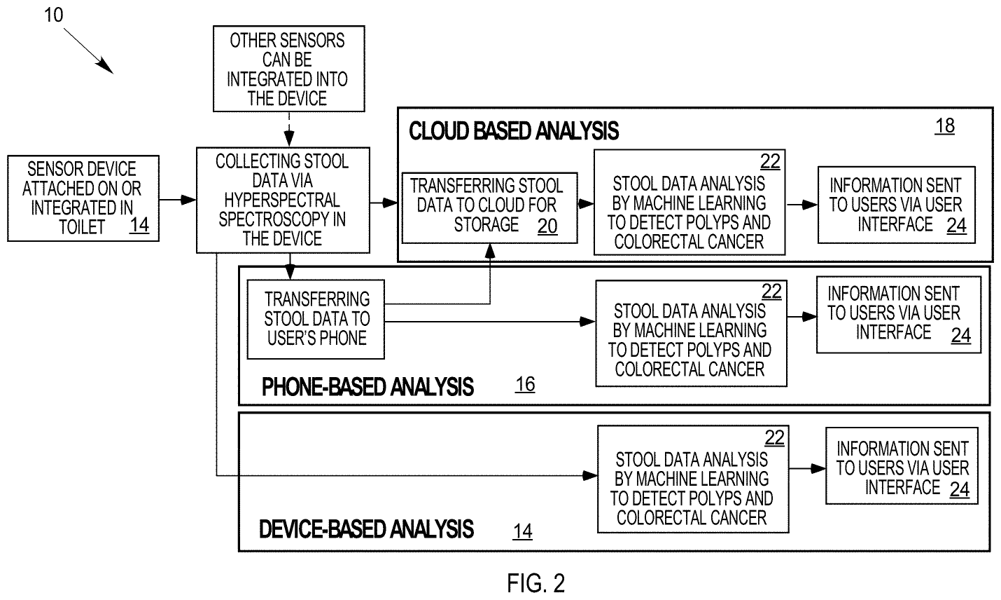

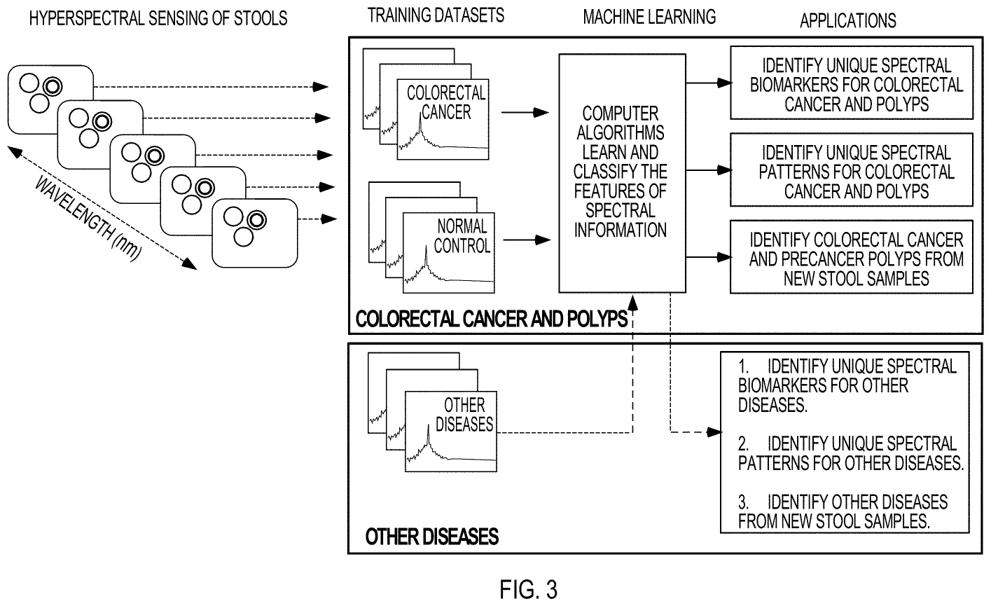

FIGS. 2 and 3 illustrate more details of portions of the system shown in FIG. 1 and specifically more details of the analysis that may be performed by the stool sample sensor 14, the computing device 16 and the backend 18. As shown in FIG. 2, the stool sample sensor device 14 may be attached to or integrated in the toilet and may have, in addition to the sensors that generate/gather the spectral data, other sensors that generate other types of data. For example, the other type of sensors include but are not limited to optical and chemical sensors that can be integrated into the stool sample sensor device 14. As shown in the embodiment in FIG. 2, the stool sample sensor device 14 may generate/collect stool data via hyperspectral spectroscopy in the device 14 that may be transferred/communicated to the computing device 16 or the backend 18 via known wireless or wired communication techniques.

FIG. 2 shows several different embodiments by which the stool sample data may be analyzed (analysis by the backend 18, analysis by the computing device 16 or analysis by the sensor device 14 or analysis by a combination of one or more of the backend 18, computing device 16 and sensor device 14.) In the backend 18 analysis (performed in the cloud as cloud-based analysis), the backend

In one embodiment, the backend 18 may be implemented using cloud based computing resources. The backend may further comprises an analysis engine that may perform artificial intelligence operations on the data from the stool samples. In one embodiment, the analysis engine may be implemented as a plurality of lines of instructions being executed by a processor that is part of the backend 18 to perform the data analysis. In one embodiment, the data analysis may be machine learning algorithms (such as classification, pattern recognition and image recognition to process our spectrum data from stool samples.) These machine learning processes may be implemented, for example, using Google Cloud's commercially available AutoML engine. When the data is being analyzed in the backend 18, the data may be stored (20) in the backend 18. Then, stool data analysis is performed by machine learning (22) to detect symptoms or conditions, such as a polyps and colorectal cancer in one embodiment. The results analysis may be sent to the users (doctor and/or patient/subject) (24) via a user interface, examples of which are shown in FIGS. 19-20.

When the computing device 16 performs the data analysis, the computing device may further comprises an analysis engine that may perform artificial intelligence operations on the data from the stool samples. In one embodiment, the analysis engine may be implemented as a plurality of lines of instructions being executed by a processor that is part of the computing device 16 to perform the data analysis. In one embodiment, the data analysis may be machine learning algorithms (such as classification, pattern recognition and image recognition to process our spectrum data from stool samples.) When the data is being analyzed in the computing device 16, the stool data analysis is performed by machine learning (22) to detect symptoms or conditions, such as a polyps and colorectal cancer in one embodiment. The results analysis may be sent to the users (doctor and/or patient/subject) (24) via a user interface, examples of which are shown in FIGS. 19-20.

When the stool sample sensor device 14 performs the data analysis, the computing device may further comprises an analysis engine that may perform artificial intelligence operations on the data from the stool samples. In one embodiment, the analysis engine may be implemented as a plurality of lines of instructions being executed by a processor that is part of the stool sample sensor device 14 to perform the data analysis. In one embodiment, the data analysis may be machine learning algorithms (such as classification, pattern recognition and image recognition to process our spectrum data from stool samples.) When the data is being analyzed in the stool sample sensor device 14, stool data analysis is performed by machine learning (22) to detect symptoms or conditions, such as a polyps and colorectal cancer in one embodiment. The results analysis may be sent to the users (doctor and/or patient/subject) (24) via a user interface, examples of which are shown in FIGS. 19-20.

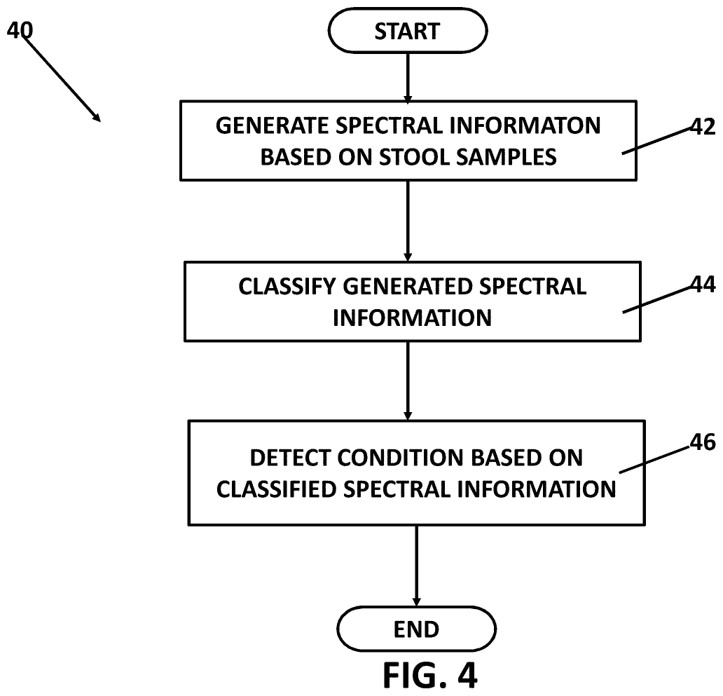

FIGS. 3 and 4 shows more details of a method 40, using the system shown in FIG. 1, to detect colorectal cancer from stool samples and/or other diseases. In the method, stool samples are collected from subjects with colorectal cancer, precancer polyps, noncancerous (normal) and those stool samples were subjected to a hyperspectral spectrometer-based sensor device 14 (42). The spectra across different wavelength bands between 200-1,000 nm were then collected by the spectrometer (the set of spectra biomarkers or the hyperspectral signal). The data gathered/collected by the hyperspectral spectrometer-based sensor device 14 for the stool samples may be used as datasets to train the analysis engine (including machine learning processes described above) to identify spectral biomarkers and spectral patterns of stool samples from colorectal cancer, precancer polyps, other diseases and the healthy subject. Thus, the analysis engine learns and classifiers the features of the spectral information (44). Then, a new set of spectral data from a new stool sample may be captured in real time by the sensor device 14 and compared to the known spectral patterns of the data analysis engine (based on the training data) (46) and identify one or more of colorectal cancer, polyps, other diseases or a heathy subject (the absence of the spectral patterns for colorectal cancer, polyps or other diseases. Based on the similarity of the spectra for the new stool sample and the spectral pattern indicative of one or more of colorectal cancer, polyps and other diseases (that can be an exact match or within a predetermined percentage of the spectral pattern), the system is able to detect that the subject has one or more of colorectal cancer, polyps and other diseases.

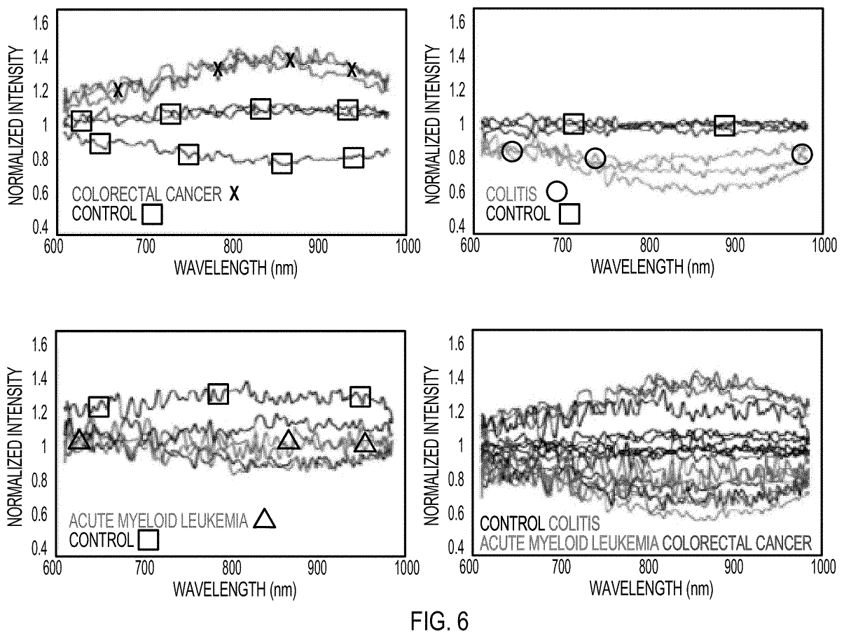

FIG. 5 illustrates an example of hyperspectral imaging analysis (using a hyperspectral imaging camera implementation for the image sensor) that can identify conditions, including colorectal cancer or polyps from the spectral data from the stool samples and FIG. 6 illustrates examples of the results of the hyperspectral imaging analysis. FIG. 5 shows the hyperspectral imaging sensor/camera in which hyperspectral images are collected at different wavelengths from the sensor and the signals from stool samples are then analyzed and classified using statistic methods or computer algorithms. FIG. 6 shows the hyperspectral imaging analysis of the stool samples from healthy controls (black, n=3 marked with square boxes) and C57Bl/6 mice with Apc^{mut}/Kras^{G12D}/p53^{mut} colorectal cancer (red, n=3 marked with Xs) showing the normalized intensity and different wavelengths and the difference in spectra for a health subject and for a subject with colorectral cancer. FIG. 6 also shows the hyperspectral imaging analysis of the stool samples from healthy controls (black, n=3 marked with square boxes) and C57Bl/6 mice with colitis (green, n=3 marked with circles) showing the normalized intensity and different wavelengths and the difference in spectra for a health subject and for a subject with colitis. FIG. 6 also shows hyperspectral imaging analysis of the stool samples from healthy controls (black, n=3 marked with square boxes) and C57Bl/6 mice with acute myeloid leukemia (blue, n=3 marked with diamonds) showing the normalized intensity and different wavelengths and the difference in spectra for a health subject and for a subject with leukemia. FIG. 6 also shows the combined results of hyperspectral imaging analysis of the healthy subjects (mice) and the mice with colorectal cancer: red; colitis: green; acute myeloid leukemia: blue. In FIG. 6, the difference of the spectral signal for the healthy subjects is due to the fact that different healthy subject were used as the control in each case.

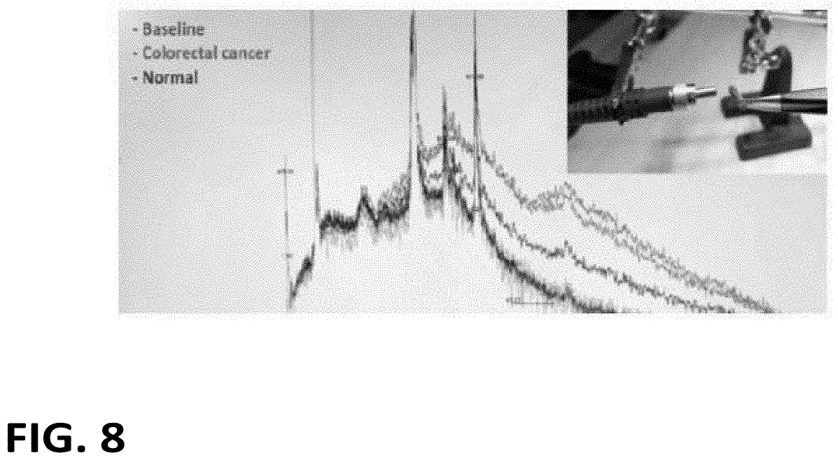

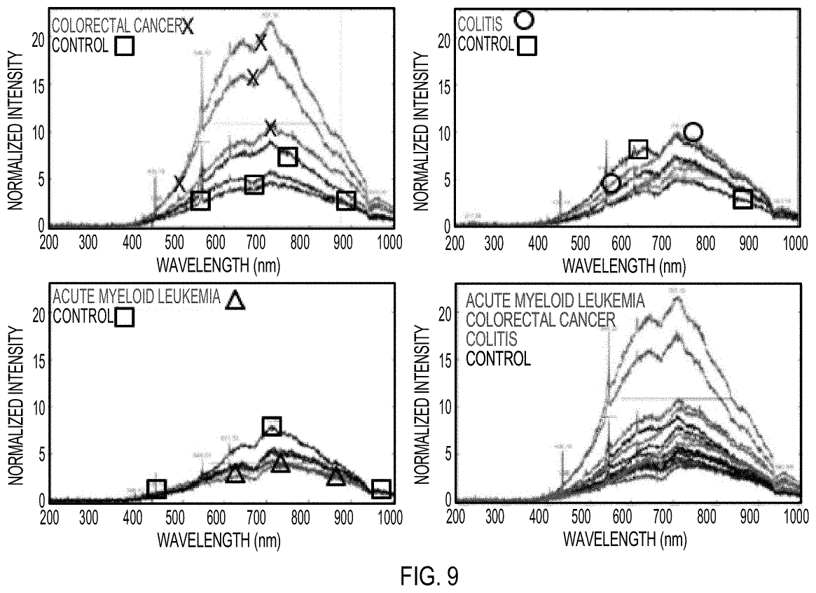

FIG. 7 illustrates an example of single point hyperspectral imaging analysis (using a single point hyperspectral imaging implementation for the image sensor) that can identify conditions, including colorectal cancer or polyps from the spectral data from the stool samples and FIGS. 8 and 9 illustrate examples of the results from the hyperspectral imaging analysis of FIG. 7. FIG. 7 shows the single point hyperspectral spectrometer in which hyperspectral images are collected at different wavelengths from the sensor and the signals from stool samples are then analyzed and classified using statistic methods or computer algorithms. FIG. 9 shows the hyperspectral imaging analysis of the stool samples from healthy controls (black, n=3 marked with square boxes) and C57Bl/6 mice with Apc^{mut}/Kras^{G12D}/p53^{mut} colorectal cancer (red, n=3 marked with Xs) showing the normalized intensity and different wavelengths and the difference in spectra for a health subject and for a subject with colorectral cancer. FIG. 9 also shows the hyperspectral imaging analysis of the stool samples from healthy controls (black, n=3 marked with square boxes) and C57Bl/6 mice with colitis (green, n=3 marked with circles) showing the normalized intensity and different wavelengths and the difference in spectra for a health subject and for a subject with colitis. FIG. 9 also shows hyperspectral imaging analysis of the stool samples from healthy controls (black, n=3 marked with square boxes) and C57Bl/6 mice with acute myeloid leukemia (red, n=3 marked with diamonds) showing the normalized intensity and different wavelengths and the difference in spectra for a health subject and for a subject with leukemia. FIG. 9 also shows the combined results of hyperspectral imaging analysis of the healthy subjects (mice) and the mice with colorectal cancer: red; colitis: green; acute myeloid leukemia: blue. In FIG. 6, the difference of the spectral signal for the healthy subjects is due to the fact that different healthy subject were used as the control in each case.

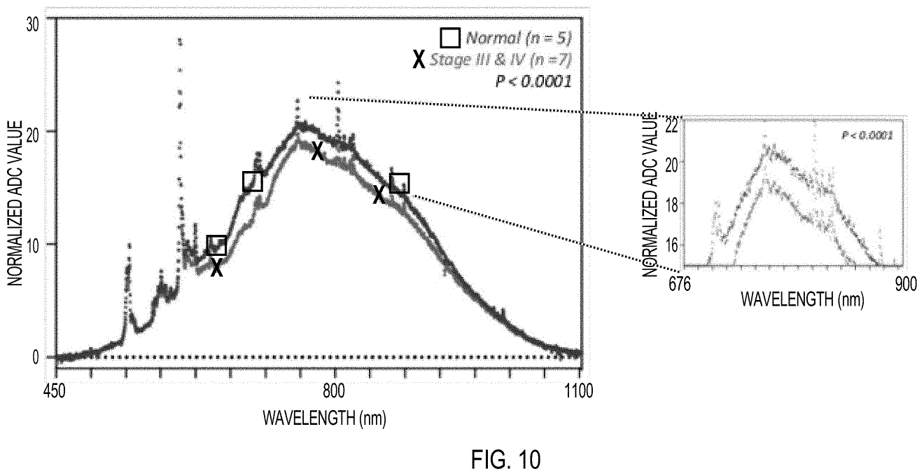

FIG. 10 illustrates the spectral analysis for later stage colorectal cancer and healthy subjects in humans based on human stool. The figure shows the normalized intensity of the spectra across different wavelength bands between 400-1,100 nm from normal human stool samples (n=5, blue line marked with square) were then classified and compared with human stool samples from colorectal cancer at stage III and IV (n=7; red marked with Xs). The observed differences in spectra from these diseases being indicative of colorectal cancer as shown in FIG. 10.

FIG. 11 illustrates the spectral analysis for polyps and stage 0 colorectal cancer and healthy subjects in humans based on human stool. FIG. 11 shows the normalized intensity of the spectra across different wavelength bands between 400-1,100 nm from normal human stool samples (n=9; blue and marked with boxes) were then classified and compared with human stool samples from precancer polyps and stage 0 (n=11; red marked with Xs) and the observed differences in spectra from these diseases being indicative of precancer polyps and stage 0.

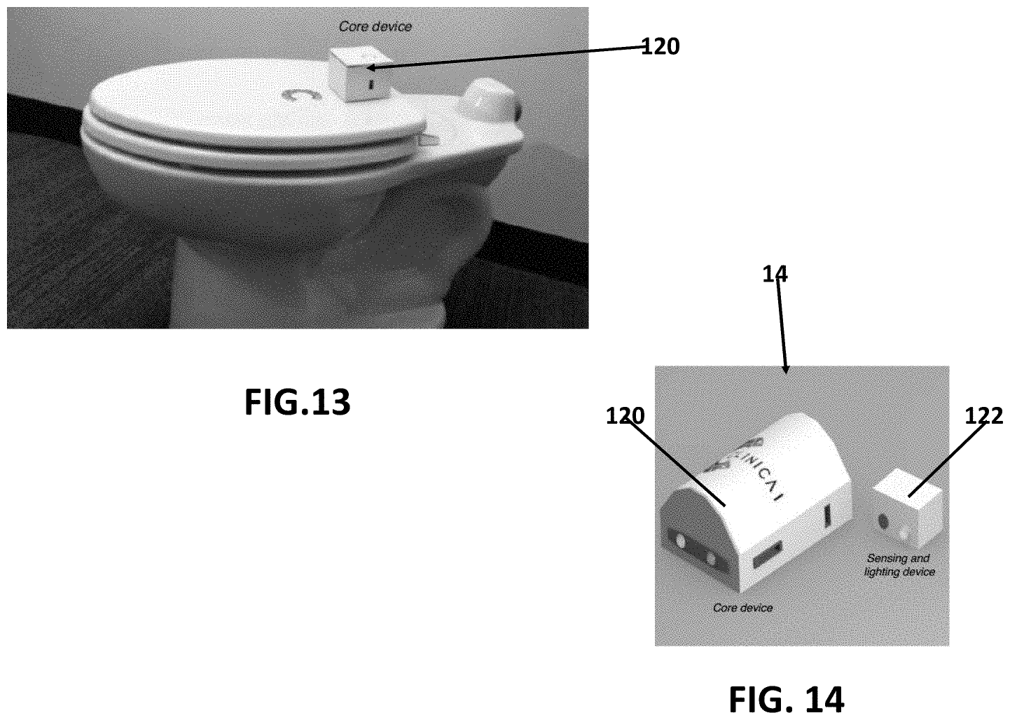

FIG. 12 illustrates an example of a first embodiment of the stool sample sensor 14 of the system in FIG. 1 and FIG. 13 illustrates the stool sample sensor 14 of FIG. 12 mounted on a toilet although the stool sample sensor 14 may be used without being mounted on a toilet in hospital environment as described above. The stool sample sensor 14 may have a core device 120 that contains the electronics, etc. of the device and a sensing/lighting device 122 that may be connected to the core device by a cable. The stool sample sensor 14 may further comprise a mounting bracket 124 that holds the sensing and lighting device 122 as shown in FIG. 2. FIG. 13 shows the core device 120 mounted on a toilet. As shown in FIG. 13, while the core device 120 may be mounted on the lid of the toilet or integrated into the toilet, the sensing and lighting device 122 may be positioned at the back of the toilet under the toilet seat that permits sensing and taking stool samples accurately, and also lighting up the good range inside toilet without shadow and the light source should cover 360-2500 nm, or any range between 360-2500 nm, or 200 to 11,111 nm.

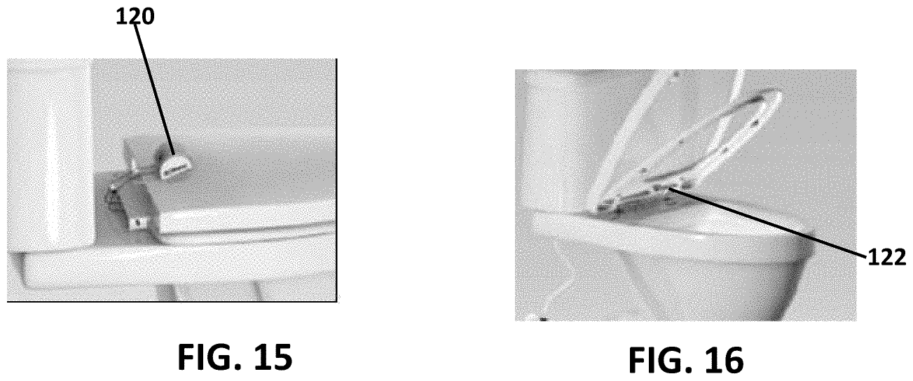

FIGS. 14-16 illustrate an example of a second embodiment of the stool sample sensor device 14 of the system in FIG. 1 and the stool sample sensor mounted on a toilet. The stool sample sensor device 14 has the same core device 120 and the sensing and lighting device 122 in which the two devices 120, 122 are connected to each other via a cable/wires that can be seen in FIG. 15. With either of the sample sensor device 14, one or both of the devices 120, 122 also may be integrated into the toilet.

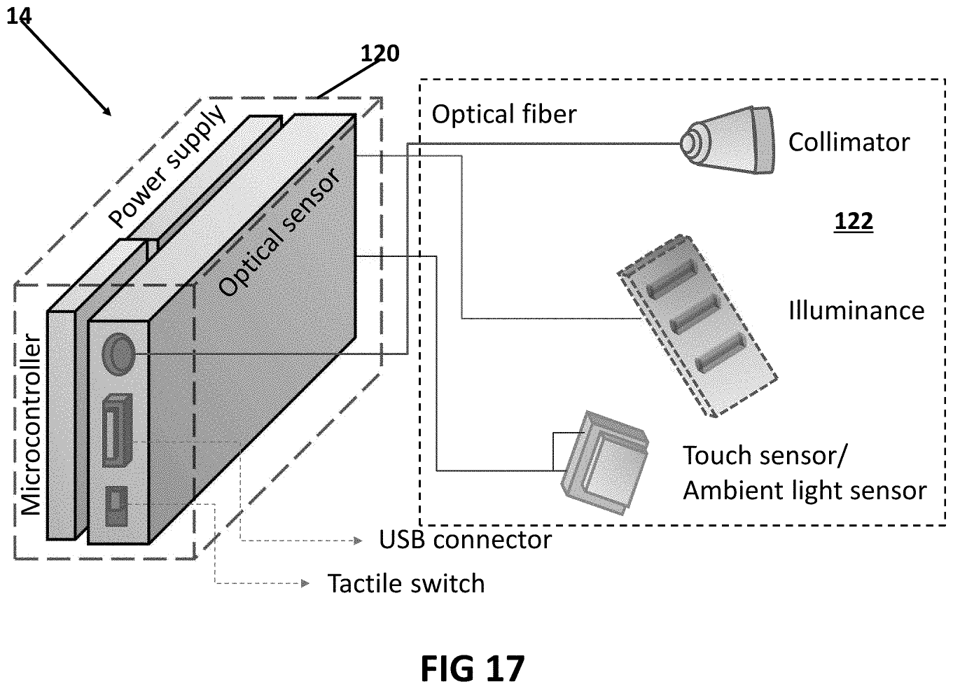

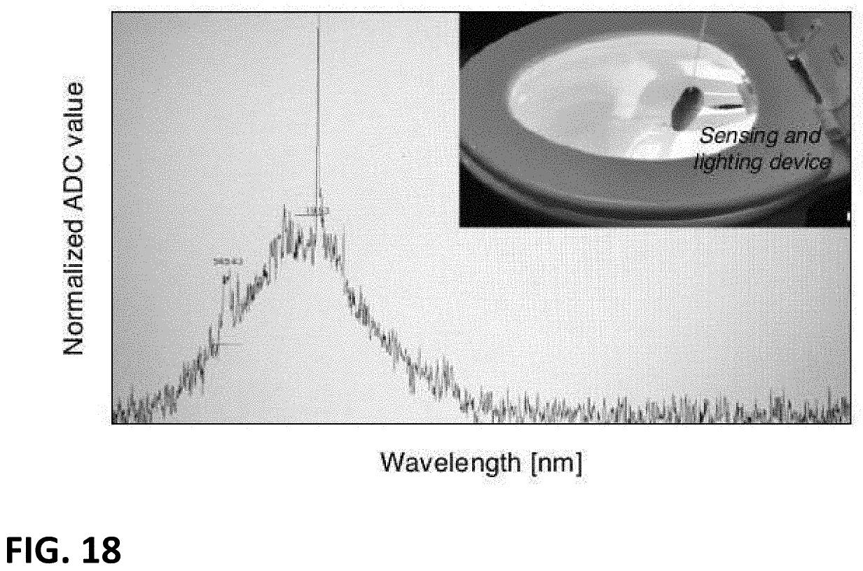

FIG. 17 illustrates more details of the core device 120 and imaging and sensing device 122 that are part of the stool sample sensor 14 and FIG. 18 illustrates an example of the spectra generated by the image sensor. As shown in FIG. 17, the core device 120 may further comprise a microcontroller, a power supply and an optical sensor that are interconnected. The optical sensor may further comprise a USB connector and a tactile switch and performs the hyperspectral spectrometer. The tactile switch is a power switch and the power supply may be rechargeable and removable so that it can be replaced with a new power supply. The power supply supplies power to the microcontroller and the optical sensor and the microcontroller controls the operation of the optical sensor, captures the spectra data from the optical sensor and prepared that spectral data for transfer when the backend or the computing device is performing is data analysis. The core device 120 further comprises a communication circuit (wireless or wired) that allows the core device 120 to communicate the stool sample spectral data to the backend or the computing device. The sensing and lighting device 122 may further comprise a collimator that is connected via an optical fiber to the optical sensor to gather the spectral data, an illuminator device that can direct illumination towards the stool sample and a touch sensor/ambient light sensor that measures environmental data such as the ambient light. The core sensor device scans an object through the collimator and the collimator can be used to couple collimated light into a multi-mode optical fiber or to collimate the divergent light emitted from a fiber. The illuminance instrument can be adjusted the light intensity. The touch sensor/ambient light sensor instrument. The purpose of this part is to control the power supply to turn on/off.

FIG. 19 illustrates an example of the user interface for a user generated by the system in FIG. 1 and FIG. 20 illustrates an example of the user interface for a doctor generated by the system in FIG. 1. For example, the user interface will display an indication of colorectal cancer, precancerous polyps and/or “presence of blood” as shown in the example user interfaces.

Method for Detecting Colorectal Cancer and Precancerous from Stool Samples

One aspect of system relates to a method for detecting colorectal cancer and precancerous polyps from stool samples. The method comprises the step of subjecting stool samples from cancerous and non-cancerous subjects to hyperspectral spectroscopy and wherein differences in spectra indicates cancer, or assesses risk of development thereof. In another aspect, the method is for identifying spectral biomarkers and patterns from stool samples from a condition, wherein the condition is selected from the group comprising: colorectal cancer, precancerous polyps, gastrointestinal diseases, inflammatory pain, chronic disease, cardiovascular diseases, cardiac hypertrophy, heart failure, cancer. The cancer may be carcinoma, sarcoma, melanoma, germ cell tumor, lymphomas or leukemia.

In some embodiments, the hyperspectral spectrometer is selected from the group comprising: spectral range between 200 to 11,111 nm, spectral range including Ultraviolet (UV) and visible spectrum and Near-infrared (NIR), spectral resolution between 1 to 10 μm/pixel, spectral resolution (Full Width at Half Maximum) between 0.3 nm to 1.5 nm. Si (Silicon) charge-coupled device (CCD) detector, charge-coupled device (CCD) detector, HgCdTe (Mercury Cadmium Telluride) detector, intensified charge-coupled device (ICCD) detector, InGaAs (Indium Gallium Arsenide) detector, focal-plane array (FPA) detector, filter wheel dispersive device, prism dispersive device, Liquid crystal tunable filters (LCTF) dispersive device, acousto-optic tunable filter (AOTF) dispersive device, grating dispersive device, computer generated holographic (CGH) dispersive device, prism-grating-prism (PGP) dispersive device, staring acquisition mode, pushbroom acquisition mode, Fourier-transform infrared spectroscopy (FTIR) acquisition mode, snapshot acquisition mode, reflectance measurement mode, fluorescence and reflectance measurement mode, transmission measurement mode, fluorescence measurement mode.

In another aspect, the application relates to a composition for detecting colorectal cancer and precancerous polyps from stool samples. The composition comprises: a hyperspectral spectrometer and a statistic method or a computer algorithm to compare the resulting hyperspectral spectrum of stool from non-cancerous and colorectal cancer subjects, and identify differences in spectra being indicative of colorectal cancer.

In another aspect, the application relates to a method for detecting colorectal cancer and precancerous polyps from stool samples without mixing the samples with buffer to extract the supernatant for analysis. The stool samples are directly subjected to hyperspectral spectroscopy, and can be used for other spectroscopy including infrared and near-infrared spectroscopy.

Method for Identifying Spectral Biomarkers and Patterns from Stool Samples from Cancerous and Inflammatory Bowel Disease Subjects

In another aspect, the application relates to a method for identifying novel spectral biomarkers and patterns from stool samples from a condition. The method comprises the steps of: (a) collecting stool samples from subjects with different conditions; (b) analyzing the stool samples by hyperspectral spectroscopy; (c) classifying spectra from stool samples from different subjects; (d) developing computer algorithms; and (e) identifying spectral biomarkers and patterns.

In some embodiments, the condition is selected from the group comprising: colorectal cancer, precancerous polyps, gastrointestinal diseases, inflammatory pain, chronic disease, cardiovascular diseases, cardiac hypertrophy, heart failure, cancer. The cancer may be carcinoma, sarcoma, melanoma, germ cell tumor, lymphomas or leukemia.

In some embodiments, the spectral biomarkers and patterns is in the range between 200 to 11,111 nm.

In some embodiments, the spectral biomarkers and patterns is in the range spectral range including Ultraviolet (UV) and visible spectrum and Near-infrared (NIR).

### EXAMPLES

### Example 1. Materials and Methods

Collection of Murine Stool Samples

The stool samples were collected from normal C57Bl/6 mice; from the C57Bl/6 mice with Apc^{mut}/Kras^{G12D}/p53^{mut} colorectal cancer tumors (O'Rourke K P et al. Nat Biotechnol. 2017); from the C57Bl/6 mice with colitis induced by Dextran Sodium Sulfate (DSS) for a week (WO2017100432A1); and from the C57Bl/6 mice with acute myeloid leukemia.

Hyperspectral Spectroscopy Analysis of Stool Samples

Stool samples collected from mice which have colorectal cancer or colitis or acute myeloid leukemia were directly subjected to hyperspectral spectroscopy without mixing the samples with buffer to extract the supernatant for analysis. Images or single point spectra across different wavelength bands between 200-1,000 nm were then collected by spectrometers and processed by computer software to extract the spectra intensities from the images. Normalized intensity of the spectra across different wavelength bands between 200-1,000 nm from healthy stool samples were then classified and compared with stool samples from colorectal cancer, colitis or acute myeloid leukemia subjects. The observed differences in spectra from these diseases being indicative of colorectal cancer.

### Example 2. Hyperspectral Spectroscopy Analyzes Stool Samples and Identifies Colorectal Cancer Subjects from Other Cancer and Colitis

Stool samples collected from mice which have colorectal cancer or colitis or acute myeloid leukemia were subjected to a hyperspectral imaging sensor. Images across different wavelength bands between 600-1,000 nm were then collected by the sensor and processed by computer software to extract the spectra intensities from the images. Normalized intensity of the spectra across different wavelength bands between 600-1,000 nm from healthy stool samples were then classified and compared with stool samples from colorectal cancer, colitis or acute myeloid leukemia subjects. The observed differences in spectra from these diseases being indicative of colorectal cancer.

Stool samples collected from mice which have colorectal cancer or colitis or acute myeloid leukemia were subjected to a single point hyperspectral spectrometer. Spectra across different wavelength bands between 200-1,000 nm were then collected by the spectrometer and processed by computer software to extract the spectra intensities. Normalized intensity of the spectra across different wavelength bands between 200-1,000 nm from healthy stool samples were then classified and compared with stool samples from colorectal cancer, colitis or acute myeloid leukemia subjects. The observed differences in spectra from these diseases being indicative of colorectal cancer.

Spectra across different wavelength bands between 200-1,000 nm collected from stool samples from colorectal cancer, colitis or acute myeloid leukemia were used as datasets to train computer algorithms by machine learning methods to identify spectral biomarkers and patterns from stool samples from cancerous and inflammatory bowel disease subjects.

### Example 3. Hyperspectral Spectroscopy Analyzes Stool Samples and Identifies Colorectal Cancer and Precancerous Polyps from Noncancerous Subjects

Stool samples collected from human which have colorectal cancer or precancerous polyps or noncancerous were subjected to a single point hyperspectral spectrometer. Spectra across different wavelength bands between 450-1,100 nm were then collected by the spectrometer and processed by computer software to extract the spectra intensities. Normalized intensity of the spectra across different wavelength bands between 450-1,100 nm from noncancerous stool samples were then classified and compared with stool samples from colorectal cancer or precancerous polyps subjects. The observed differences in spectra from these diseases being indicative of colorectal cancer or precancerous polyps.

Spectra across different wavelength bands between 450-1,100 nm collected from stool samples from colorectal cancer or precancerous polyps or noncancerous were used as datasets to train computer algorithms by machine learning methods to identify spectral biomarkers and patterns from stool samples from colorectal cancer or precancerous polyps subjects.

What is claimed is:

1. A method for detecting colorectal cancer and precancerous polyps, comprising:
capturing, using an image sensor, hyperspectral spectra from a stool sample of a subject;
comparing, using an analysis engine connected to the image sensor, the hyperspectral spectra from the stool sample to a spectral pattern indicative of colorectal cancer to identify a similarity between the hyperspectral spectra from the stool sample to the spectral pattern indicative of colorectal cancer; and
detecting colorectal cancer of the subject when there is a high similarity between the hyperspectral spectra from the stool sample to the spectral pattern indicative of colorectal cancer.

2. The method of claim 1 further comprising gathering spectral signals using the image sensor from a stool sample of a subject that has cancer and from a stool sample of a healthy subject and training the analysis engine using the spectral signals of the subject with cancer and the healthy subjects to generate the spectral pattern indicative of colorectal cancer.

3. The method of claim 2, wherein comparing the hyperspectral spectra from the stool sample to the spectral pattern indicative of colorectal cancer further comprises performing machine learning to generate the similarity between the hyperspectral spectra from the stool sample to the spectral pattern indicative of colorectal cancer.

4. The method of claim 1, wherein capturing the hyperspectral spectra from a stool sample of a subject further comprises capturing the hyperspectral spectra while the stool sample is in a toilet and without mixing the stool sample with a buffer.

5. The method of claim 1, wherein capturing the hyperspectral spectra from a stool sample of a subject further comprises using a hyperspectral camera or a single point hyperspectral spectroscopy device to capture the hyperspectral spectra from a stool sample of a subject.

6. The method of claim 5, wherein capturing the hyperspectral spectra from a stool sample of a subject further comprises collecting and processing images and spectral information between 200 to 11,111 nm.

7. The method of claim 5, wherein capturing the hyperspectral spectra from a stool sample of a subject further comprises collecting and processing images and spectral information from Ultraviolet (UV), visible spectrum and Near-infrared (NIR).

8. A method for identifying spectral biomarkers and patterns from stool samples, comprising the steps of:
capturing, using an image sensor, hyperspectral spectra from a stool sample of a subject;
comparing, using an analysis engine connected to the image sensor, the hyperspectral spectra from the stool sample to a spectral pattern indicative of cancer or a spectral pattern indicative of inflammatory bowel disease to identify a similarity between the hyperspectral spectra from the stool sample to the spectral pattern indicative of cancer or the spectral pattern indicative of inflammatory bowel disease; and
detecting one of cancer and inflammatory bowel disease of the subject when there is a high similarity between the hyperspectral spectra from the stool sample to the spectral pattern indicative of cancer or the spectral pattern indicative of inflammatory bowel disease.

9. The method of claim 8 further comprising gathering spectral signals using the image sensor from a stool sample of a subject that has cancer, from a stool sample of a subject that has inflammatory bowel disease and from a stool sample of a healthy subject and training the analysis engine using the spectral signals of the subject with cancer, the spectral signals of the subject with inflammatory bowel disease and the healthy subjects to generate the spectral pattern indicative of cancer and the spectral pattern indicative of inflammatory bowel disease.

10. The method of claim 9, wherein comparing the hyperspectral spectra from the stool sample to the spectral patterns indicative of cancer and inflammatory bowel disease further comprises performing machine learning to generate the similarity between the hyperspectral spectra from the stool sample to the spectral patterns indicative of cancer and inflammatory bowel disease.

11. The method of claim 8, wherein capturing the hyperspectral spectra from a stool sample of a subject further comprises capturing the hyperspectral spectra while the stool sample is in a toilet and without mixing the stool sample with a buffer.

12. The method of claim 8, wherein capturing the hyperspectral spectra from a stool sample of a subject further comprises using a hyperspectral camera or a single point hyperspectral spectroscopy device to capture the hyperspectral spectra from a stool sample of a subject.

13. The method of claim 8, wherein the cancer includes one or more of carcinomas, leukemia, lymphoma, sarcomas, melanoma and germ cell tumors subjects.

14. The method of claim 12, wherein capturing the hyperspectral spectra from a stool sample of a subject further comprises collecting and processing images and spectral information between 200 to 11,111 nm.

15. The method of claim 12, wherein capturing the hyperspectral spectra from a stool sample of a subject further comprises collecting and processing images and spectral information from Ultraviolet (UV), visible spectrum and Near-infrared (NIR).

16. An apparatus for detecting colorectal cancer and precancerous polyps, comprising:
an image sensor that captures hyperspectral spectra from a stool sample of a subject;
an analysis engine connected to the image sensor that compares the hyperspectral spectra from the stool sample to a spectral pattern indicative of colorectal cancer to identify a similarity between the hyperspectral spectra from the stool sample to the spectral pattern indicative of colorectal cancer; and
a computing device having a display that indicates colorectal cancer of the subject when there is a high similarity between the hyperspectral spectra from the stool sample to the spectral pattern indicative of colorectal cancer.

17. The apparatus of claim 16, wherein the image sensor gathers spectral signals using the image sensor from a stool sample of a subject that has cancer and from a stool sample of a healthy subject and wherein the analysis engine is trained using the spectral signals of the subject with cancer and the healthy subjects to generate the spectral pattern indicative of colorectal cancer.

18. The apparatus of claim 17, wherein the analysis engine performs machine learning to generate the similarity between the hyperspectral spectra from the stool sample to the spectral pattern indicative of colorectal cancer.

19. The apparatus of claim 16, wherein the image sensor captures the hyperspectral spectra while the stool sample is in a toilet and without mixing the stool sample with a buffer.

20. The apparatus of claim 16, wherein the image sensor is one of a hyperspectral camera and a single point hyperspectral spectroscopy device.

21. The apparatus of claim 20, wherein the image sensor captures images and spectral information between 200 to 11,111 nm.

22. The apparatus of claim 20, wherein the image sensor captures Ultraviolet (UV), visible spectrum and Near-infrared (NIR).

23. The apparatus of claim 16 further comprising a toilet and wherein the image sensor is mounted on the toilet.

24. An apparatus for identifying spectral biomarkers and patterns from stool samples, comprising the steps of:
an image sensor that captures hyperspectral spectra from a stool sample of a subject;
an analysis engine connected to the image sensor that compares the hyperspectral spectra from the stool sample to a spectral pattern indicative of cancer or a spectral pattern indicative of inflammatory bowel disease to identify a similarity between the hyperspectral spectra from the stool sample to the spectral pattern indicative of cancer or the spectral pattern indicative of inflammatory bowel disease; and
a computing device having a display that displays an indication of cancer and inflammatory bowel disease of the subject when there is a high similarity between the hyperspectral spectra from the stool sample to the spectral pattern indicative of cancer or the spectral pattern indicative of inflammatory bowel disease.

25. The apparatus of claim 24, wherein the image sensor gathers spectral signals using the image sensor from a stool sample of a subject that has cancer, from a stool sample of a subject that has inflammatory bowel disease and from a stool sample of a healthy subject and wherein the analysis engine is trained using the spectral signals of the subject with cancer and inflammatory bowel disease and the healthy subjects to generate the spectral pattern indicative of cancer and inflammatory bowel disease.

26. The apparatus of claim 25, wherein the analysis engine performs machine learning to generate the similarity between the hyperspectral spectra from the stool sample to the spectral pattern indicative of cancer or inflammatory bowel disease.

27. The apparatus of claim 24, wherein the image sensor captures the hyperspectral spectra while the stool sample is in a toilet and without mixing the stool sample with a buffer.

28. The apparatus of claim 24, wherein the image sensor is one of a hyperspectral camera and a single point hyperspectral spectroscopy device.

29. The method of claim 24, wherein the cancer includes one or more of carcinomas, leukemia, lymphoma, sarcomas, melanoma and germ cell tumors subjects.

30. The apparatus of claim 28, wherein the image sensor captures images and spectral information between 200 to 11,111 nm.

31. The apparatus of claim 28, wherein the image sensor captures Ultraviolet (UV), visible spectrum and Near-infrared (NIR).

32. The apparatus of claim 24 further comprising a toilet and wherein the image sensor is mounted on the toilet.
